(19) United States
(12) Patent Application Publication
(10) Pub. No.: US 2018/0078148 A1
(43) Pub. Date: Mar. 22, 2018

(54) BIOIMPEDANCE BASED PULSE WAVEFORM SENSING

(71) Applicant: Microsoft Technology Licensing, LLC, Redmond, WA (US)

(72) Inventors: Gabriel Adam COHN, Sammamish, WA (US); Roman KUSCHE, Hamburg (DE)

(73) Assignee: Microsoft Technology Licensing, LLC, Redmond, WA (US)

(21) Appl. No.: 15/620,651

(22) Filed: Jun. 12, 2017

Related U.S. Application Data
(60) Provisional application No. 62/398,331, filed on Sep. 22, 2016.

(51) Int. Cl. A61B 5/022; A61B 5/00; A61B 5/053

(52) U.S. Cl. CPC A61B 5/022; A61B 5/6824; A61B 5/7289; A61B 5/7435; A61B 5/0531

(57) ABSTRACT

An arterial pulse wave may be determined via a system that includes a pressure transducing pad configured to temporarily attach to skin of a user and to deflect outwards from the skin proportionate to pressure applied by an artery. A sensor is configured to measure outward deflection of the pressure transducing pad. A plurality of electrodes are coupled to the pressure transducing pad and configured to interface with the skin of the user when the pressure transducing pad is attached to the skin of the user. The plurality of electrodes include electrodes configured to apply a current to the skin of the user, and electrodes configured to measure a voltage differential across the skin of the user. An arterial pulse wave may be determined based on at least the measured outward deflection and the measured voltage differential.

### CROSS REFERENCE TO RELATED APPLICATIONS

This application claims priority to U.S. Provisional Application No. 62/398,331, filed Sep. 22, 2016, the entirety of which is hereby incorporated herein by reference.

### BACKGROUND

Monitoring heart rate, heart rate variability, arterial blood pressure, pulse-wave velocity, and augmentation index provides useful health information. These traits can be determined non-invasively based on the morphology of an arterial pulse waveform. A pulse waveform sensor may be incorporated into a wearable device.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

An arterial pulse wave may be determined via a system that includes a pressure transducing pad configured to temporarily attach to skin of a user and to deflect outwards from the skin proportionate to pressure applied by an artery. A sensor is configured to measure outward deflection of the pressure transducing pad. A plurality of electrodes are coupled to the pressure transducing pad and configured to interface with the skin of the user when the pressure transducing pad is attached to the skin of the user. The plurality of electrodes include electrodes configured to apply a current to the skin of the user, and electrodes configured to measure a voltage differential across the skin of the user. An arterial pulse wave may be determined based on at least the measured outward deflection and the measured voltage differential.

### BRIEF DESCRIPTION OF THE DRAWINGS

FIG. 1 illustrates an arm of a user with example thin-strip bioimpedance electrodes placed at the radial artery.

FIG. 2 illustrates an arm of a user with example round bioimpedance electrodes placed at the radial artery.

FIG. 3 illustrates an arm of a user with example thin-strip bioimpedance electrodes placed across the radial and ulnar arteries.

FIG. 4A shows a wearable assembly for a pulse waveform sensor.

FIG. 4B shows a cross section of a wrist of a user wearing the pulse waveform sensor of FIG. 4A.

FIG. 5 shows a graph of example data generated using the system of FIGS. 4A-4B.

FIG. 6 shows an example method for determining an arterial pulse wave of a user.

FIG. 7 shows an exploded view of a wearable electronic device comprising a pulse waveform sensor.

FIG. 8 schematically shows a sensory-and-logic system usable to transduce a pressure wave from a radial artery to a non-invasive pulse sensor.

### DETAILED DESCRIPTION

Continuous cardiac monitoring of healthy and unhealthy patients can provide information related to the progression of heart disease and enable early treatment. The morphology and velocity of the pulse wave in the arteries provide meaningful information about the cardiovascular system. Pressure-based sensing of the pulse wave offers a non-invasive approach to extracting important cardiovascular parameters, such as heart rate, augmentation index, and pulse wave velocity. Such parameters may be utilized to determine arterial stiffness, for example.

However, current non-invasive pulse-pressure sensing techniques are focused on expert clinicians employing a handheld instrument on a superficial artery (e.g., radial, carotid, femoral) or a common blood pressure cuff. While this approach can work in the clinic or lab, it may cause discomfort to the subject, and only allows for a snapshot of a person's cardiovascular state.

Recent work has explored the use of a portable sensing device worn continuously over an artery (e.g., the radial artery at the wrist) in a similar fashion to a piece of clothing or jewelry (e.g., a watch). To achieve pulse-pressure sensing in a wearable device, a physical apparatus is needed for transducing pressure waves from an artery to a non-invasive pulse-pressure sensor. However, the artery and sensor may move relative to one another over time, making calibration and achievement of consistent results challenging. Restricting movement of the sensor and/or wrist-wearable device relative to the wearer may result in a device that is uncomfortable to wear.

Accordingly, the inventors have recognized these and other problems and disclose an alternate method for determining an arterial pulse waveform that uses a bioimpedance measurement device. The bioimpedance measurement device may be used to extract important cardiovascular parameters, such as heart rate, augmentation index, and pulse wave velocity, similar to using a pressure transducer. Additionally, the bioimpedance measurement device may be used in tandem with a pressure transducer. Using both sensing approaches in tandem can provide more information than using either approach alone, and the cooperative use of both approaches may then be utilized in extracting additional important cardiovascular parameters.

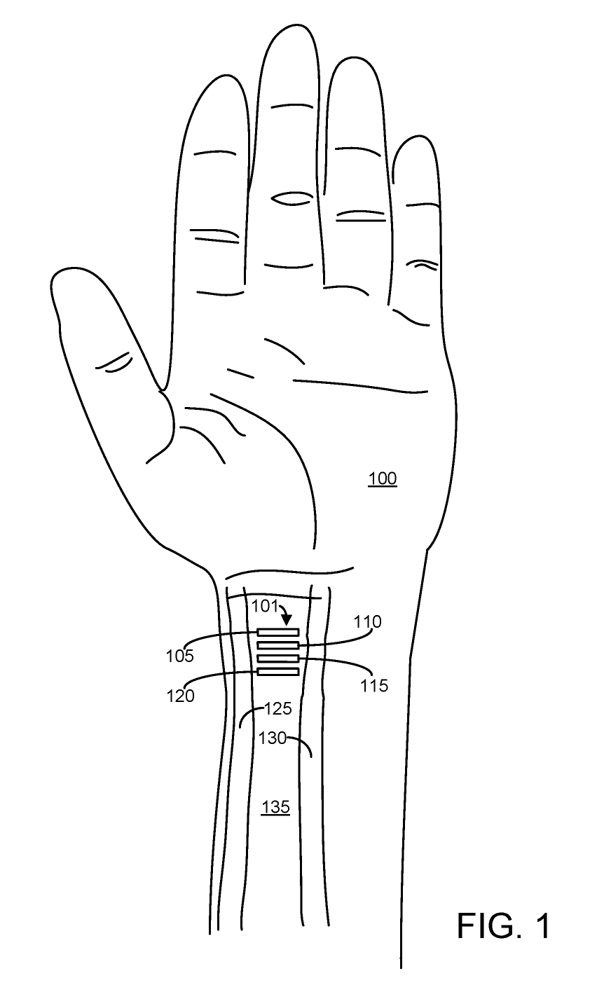

FIG. 1 illustrates an arm of a user 100 with a plurality of electrodes 101 that may be placed on the underside of the arm above an artery in order to determine a pulse waveform of user 100 via bioimpedance analysis. In bioimpedance analysis, a small alternating current (AC) is injected into the area of interest using a first pair of electrodes in order to determine the electrical impedance of body tissue. The voltage drop across the area of interest is then measured using a second pair of electrodes in order to determine the magnitude and phase of the bioimpedance. In some examples, a single pair of electrodes may perform both functions. For any cross-section of tissue, the bioimpedance is dominated by the fluid filled parts of the tissue. For this reason, bioimpedance is well suited for measuring properties of blood vessels.

In this example, four electrodes (105, 110, 115, & 120) are shown. However, in some examples fewer (e.g., 2) electrodes may be used, and in some examples more (e.g., 6) electrodes may be used. In 4-wire bioimpedance approaches, the outer two electrodes carry the current while the inner two electrodes measure the voltage in the area of interest. As such, outer electrodes 105 and 120 may be current-carrying electrodes, while inner electrodes 110 and 115 may be voltage measuring electrodes. Electrodes 105, 110, 115, and 120 may be placed on the skin of user 100 at the underside of the wrist between the radius 125 and flexor carpi radialis tendon 130 and thus may be situated above arterial lumen 135 where the radial artery is closest to the surface of the skin. In some examples, the electrodes may be self-adhering or may be secured in place with a temporary adhesive. In some examples, the electrodes may be included in a wearable assembly, such as a device with a wrist-watch or cuff type form. While most examples herein are described with regards to the radial artery, configurations where the electrodes are placed on the underside of the wrist at the ulnar artery are also possible.

Typically, bioimpedance measurements are done on relatively large areas of the body in order to obtain a constant current flux across the skin. As such, bioimpedance measurements performed on the arm typically space the electrodes across the majority of the forearm. However, to the first order, an increase in the volume or cross section of a blood vessel is manifest in a decrease in the local bioimpedance, as the increased volume of blood within the vessel makes the tissue being probed more conductive than the surrounding tissue. The total change in blood volume is relatively small, and thus the changes in bioimpedance are relatively small. This necessitates placing the electrodes such that the volume being sensed includes the blood vessel(s), but only includes a minimal amount of the surrounding tissue. In this way, changes in bioimpedance due to changes in blood volume may be isolated from noise generated by total tissue bioimpedance.

In the configuration shown in FIG. 1, wherein the electrodes are thin strips placed parallel to each other, but perpendicular to the radial artery, the bioimpedance is measured only at the radial artery, thus keeping signal quality high. With a tighter spacing across the forearm, the current injected by electrodes 105 and 120 does not penetrate the tissue as much as it would were the electrodes spaced further apart across the forearm. In this way, the percentage of the measured bioimpedance signal that is a function of blood volume within the underlying artery is increased. As such, bioimpedance analysis of a current applied directly to the radial artery yields a pulse waveform signal that is very similar to a pulse-pressure waveform measured using a tonometer or pressure sensor pressed into the radial artery.

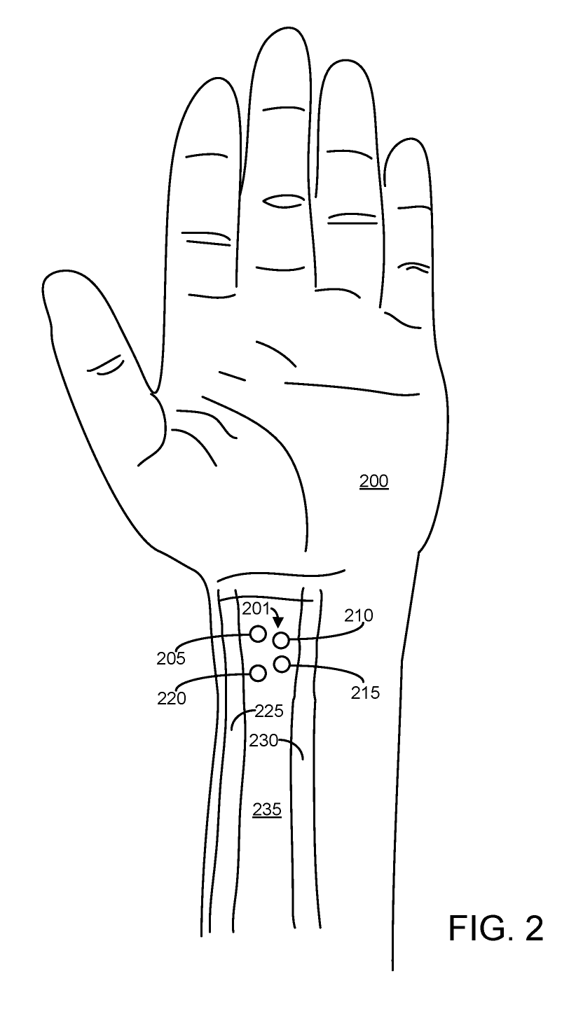

Other electrode types and configurations may be used in determining pulse waveforms via bioimpedance measurements at an artery of a user. FIG. 2 illustrates an arm of a user 200 with a plurality of electrodes 201 that may be placed on the underside of the arm above an artery in order to determine a pulse waveform of user 200 via bioimpedance analysis. Electrodes 201 include 4 round electrodes (205, 210, 215, & 220) arranged in a four-corner pattern. Outer electrodes 205 and 220 may be current-carrying electrodes, while inner electrodes 210 and 215 may be voltage measuring electrodes, though other configurations are possible. Electrodes 205, 210, 215, and 220 may be placed on the skin of user 200 at the underside of the wrist between the radius 225 and flexor carpi radialis tendon 230 and thus may be situated above radial arterial lumen 235. However, in other examples, electrodes 205, 210, 215, and 220 may be situated above the ulnar arterial lumen.

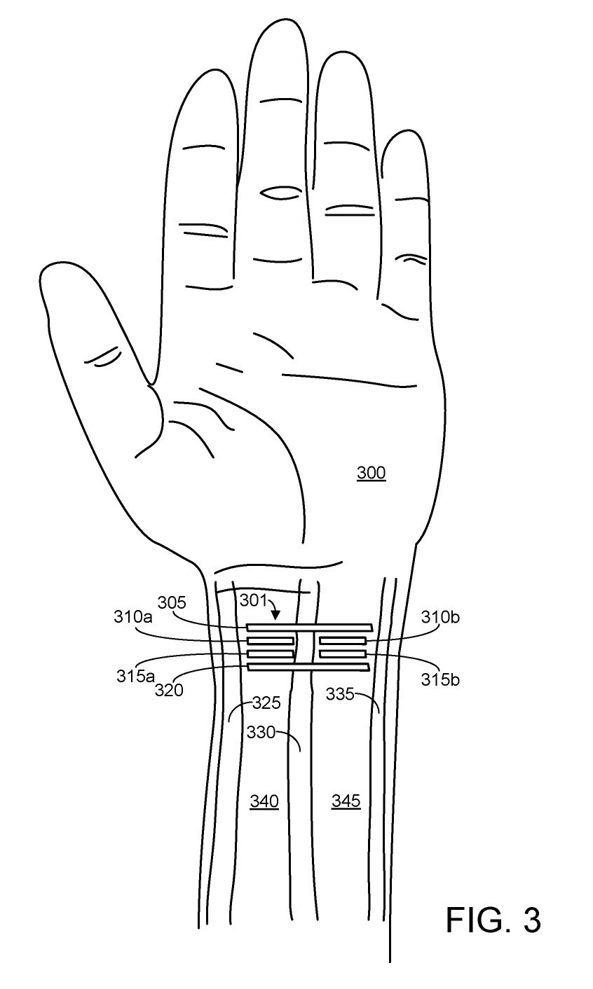

FIG. 3 illustrates an arm of a user 300 with a plurality of electrodes 301 that may be placed on the underside of the arm above an artery in order to determine a pulse waveform of user 300 via bioimpedance analysis. Electrodes 301 include 6 thin-strip electrodes (305, 310a, 310b, 315a, 315b, & 320) arranged in parallel to each other, but perpendicular to the radial and ulnar arteries. Outer electrodes 305 and 320 may be current-carrying electrodes, while inner electrodes 310a, 310b, 315a, and 315b may be voltage measuring electrodes, though other configurations are possible. In this example, electrodes 305 and 320 are placed on the skin of user 300 at the underside of the wrist, extending from the radius 325 to ulna 330 across flexor carpi radialis tendon 335, and thus may be situated above both radial arterial lumen 340 and ulnar arterial lumen 345. Electrodes 310a and 315a may be placed on the skin of user 300 at the underside of the wrist between the radius 325 and flexor carpi radialis tendon 330 and thus may be situated above radial arterial lumen 340. Electrodes 310b and 315b may be placed on the skin of user 300 at the underside of the wrist between the ulna 335 and flexor carpi radialis tendon 330 and thus may be situated above ulnar arterial lumen 345.

Several different electrode materials may be used, such as metal (e.g., silver chloride), conductive-gel, conductive hydrogel, metal-doped rubber, metal-doped polymer, carbon-doped rubber, and carbon-doped polymer. The electrodes may be coupled to metallic electrode interfaces which may facilitate the injection of current and the measurement of voltage.

Sensing logic and/or other hardware may be specifically tuned for measuring the radial and/or ulnar pulse wave. For example, the frequency of the injected current affects both the signal amplitude as well as the impedance between the skin and electrodes. Both frequency and current of the excitation signal may be chosen to balance signal quality and power consumption, while keeping well within safety and regulatory limits. Excitation frequencies may be used ranging from tens of kHz to several hundred kHz. One implementation uses 100 kHz, though slightly higher frequencies may yield a higher fidelity signal. One excitation current implementation uses about 650 μA_{RMS}, though larger currents may be used up to the safety/regulatory limits indicated for a particular excitation frequency. Smaller currents may also be used, though this may result in a reduced signal amplitude.

In addition to using bioimpedance analysis to measure the pulse wave at the radial artery, a pulse waveform detection device may combine the bioimpedance sensor with a pressure sensor. In some examples, both sensors are placed at the radial (or ulnar) artery, however, in other examples one sensor may be placed at the radial artery while the other sensor may be placed at the ulnar artery.

As one example, a piezo-resistive sensor may be used to measure pulse-pressure, though other sensor types are also possible. The pressure sensor may comprise a pad placed between the skin and the sensor to help transduce radial pressure signals. In one example, a plurality of bioimpedance electrodes are integrated into the same pressure pad used to transduce the arterial pressure so that both the bioimpedance and pressure may sense the exact same region of the artery. Using this hardware setup, pulse-pressure waves and pulse-bioimpedance waves may be measured simultaneously. When extracting cardiovascular parameters, both the pressure and bioimpedance waveforms can be used to provide more information than is available in either one independently.

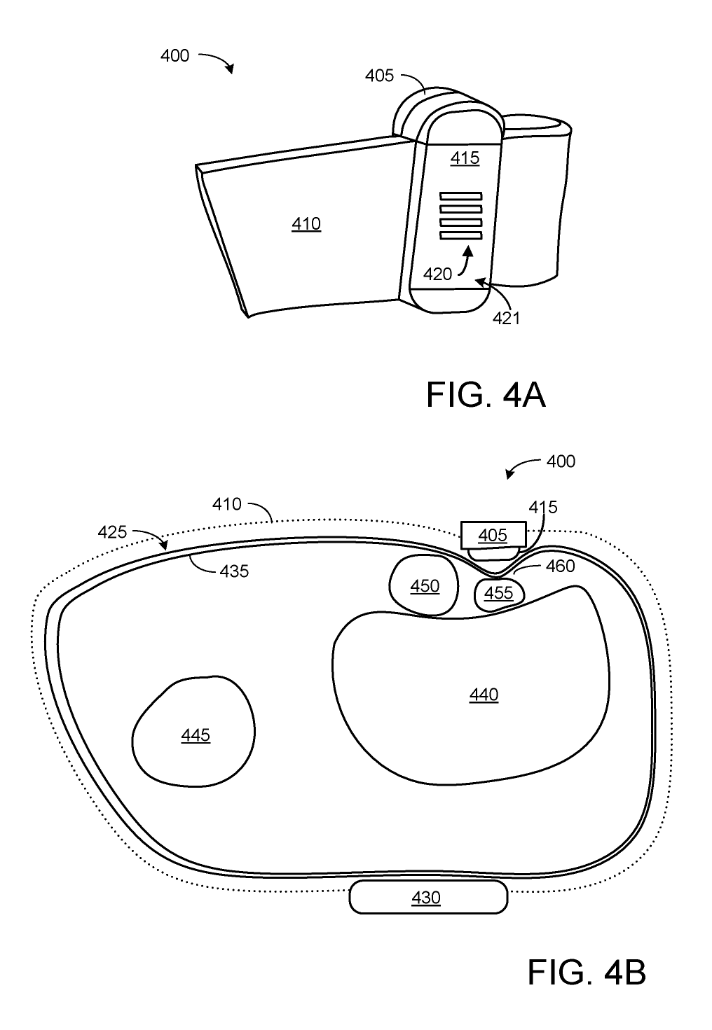

FIG. 4A shows a wearable system 400 for a pulse waveform sensor. Wearable system 400 includes a satellite housing 405 wherein a pulse waveform sensor assembly may be housed. Satellite housing 405 is coupled to strap 410, which may be used to secure the wearable assembly around a wrist of a user. Satellite housing 405 includes a pressure transducing pad 415 that may be temporarily attachable to skin of a user, and may be configured to interface with the skin of a user when wearable system is coupled to the wrist of the user. Wearable assembly 400 may include fastening componentry (not shown) to temporarily couple wearable assembly 400 to a wrist of the user, the fastening componentry configured to couple pressure transducing pad 415 in place at an artery, such as the radial or ulnar artery.

Pressure transducing pad 415 includes a plurality of electrodes 420 which may be used to measure bioimpedance of a user wearing wearable assembly 400. For example, a plurality of electrodes 420 may be coupled to an interfacing side 421 of pressure transducing pad 415 and configured to contact the skin of the user when interfacing side 421 of pressure transducing pad 415 is interfacing with the skin of the user. Plurality of electrodes 420 may protrude outward from the interfacing side of the pressure transducing pad.

Interfacing side 421 of pressure transducing pad 415 may be configured to deflect outwards from the skin proportionate to pressure applied by an artery. As such, the pressure transducing pad may be considered to be a flexible pad. In some examples, pressure transducing pad 415 may contain and/or be coupled to a pressure transducing medium to which pressure is applied proportionate to deflection of interfacing side 421. Pressure transducing pad 415 may be coupled to a pressure sensor (not shown) located within satellite housing 405, such as a piezo-electric pressure transducer. The pressure sensor may be configured to measure outward deflection of the pressure transducing pad. As an example, the pressure sensor may be a piezo-electric pressure transducer.

Plurality of electrodes 420 may comprise two electrodes configured to apply a current to the skin of the user, and further comprise two electrodes configured to measure a voltage differential across the skin of the user. As shown, plurality of electrodes 420 are thin strip electrodes arranged in parallel such that the two electrodes configured to apply a current to the skin of the user are outside of the two electrodes configured to measure a voltage differential across the skin of the user. However, as shown in FIG. 2, plurality of electrodes 420 may be round electrodes (e.g., arranged in a four-corner pattern). In some examples, plurality of electrodes 420 may comprise two electrodes configured to both apply a current to the skin of the user and to measure a voltage differential across the skin of the user.

As an example, the entire area covered by plurality of electrodes 420 may be less than or equal to a square with dimensions of 3 cm×3 cm. Each electrode may be a strip with an area less than 1 cm×2 cm. However, other dimensions and configurations may also be used.

FIG. 4B shows a cross section of wearable system 400 coupled to a wrist 425 of a user. In this example, wearable system 400 further includes primary device 430. Illustrated components of wrist 425 include skin 435, radius 440, ulna 445, flexor carpi radialis tendon 450, radial artery 455, and tissue 460. Satellite housing 405 may be placed such that pressure transducing pad 415 depresses the skin 435 of the wearer into tissue 460 between tendon 450 and radius 440, thus compressing the lumen of the radial artery. In this position, pressure pulse waves in the radial artery may apply a pressure to flexible pad 415, which may be mechanically conducted to the underlying pressure transducer via a pressure-transducing medium. Further, changes in fluid volume within radial artery 455 may impact the bioimpedance of skin 435 and tissue 460. These bioimpedance changes may be measured via electrodes 420 as described above. As electrodes 420 are subject to the same pulse pressure waves as the interfacing side 421 of pressure transducing pad 415, electrodes 420 may also deflect away from the skin in proportion to changes in pulse pressure.

A controller may be configured to output a pulse wave of the user based on at least a measured voltage differential and the measured deflection. As depicted in FIG. 4B, the voltage differential and the measured deflection may both be derived from a same artery when wearable system 400 is coupled to the wrist of the user. However, in some examples, the pressure transducing pad 415 is configured to extend across the radial artery and the ulnar artery when wearable system 400 is coupled to the wrist of the user (see for example electrodes 301 depicted in FIG. 3).

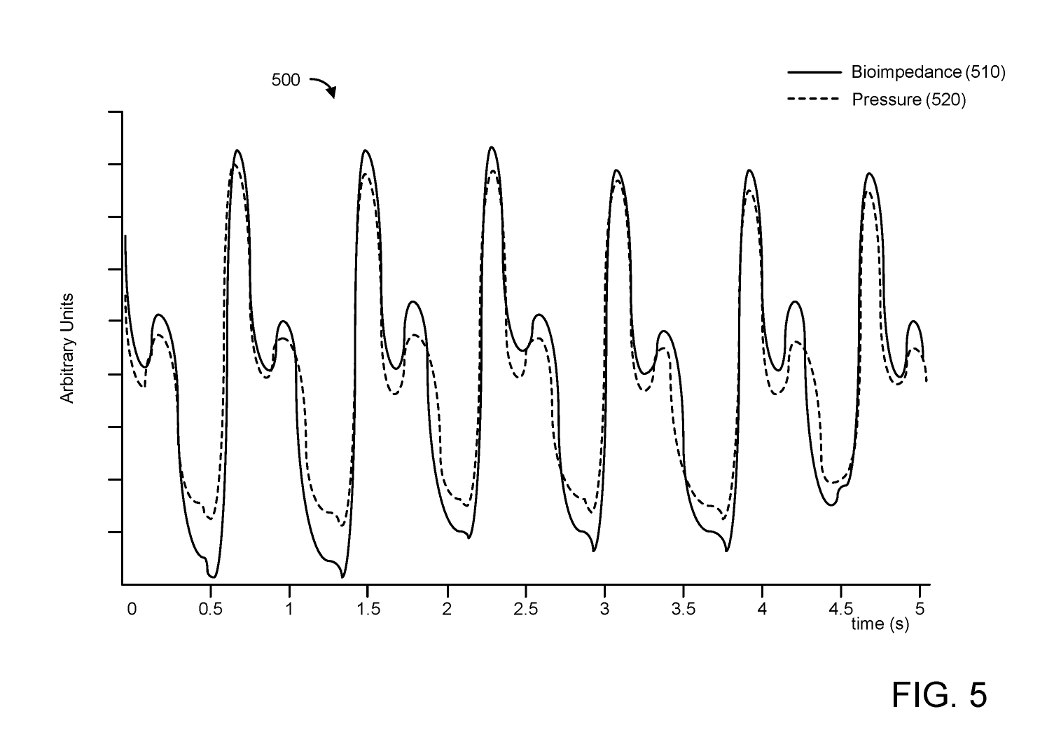

FIG. 5 shows a graph 500 of example data generated using the system of FIGS. 4A-4B. Graph 500 includes plot 510 indicating bioimpedance at a radial artery over time, and further includes plot 520, indicating pressure at the same radial artery over time. Plots 510 and 520 have been filtered and normalized so as to display over similar magnitudes. As shown in graph 500, the pulse pressure waves generated by the bioimpedance measurement device and the pulse pressure sensor have a similar morphology (period, frequency response, features, etc.). However, some higher order frequency data may be derived from the bioimpedance measurements that are not available from the pulse pressure measurements. Further, the noise produced by motion of the user may be reduced for bioimpedance measurements as compared to pulse pressure measurements.

Pulse-pressure waves provide information on internal forces pushing out of the artery, while bioimpedance is first order representative of the volume of the artery. Pressure and volume can thus provide additional information on arterial compliance and stiffness, and thus give insight into dynamic changes in cardiovascular state in addition to tracking long-term changes in arterial health over time. In this way, a greater understanding of cardiovascular function and health may be obtained than with either bioimpedance or pressure measurements alone.

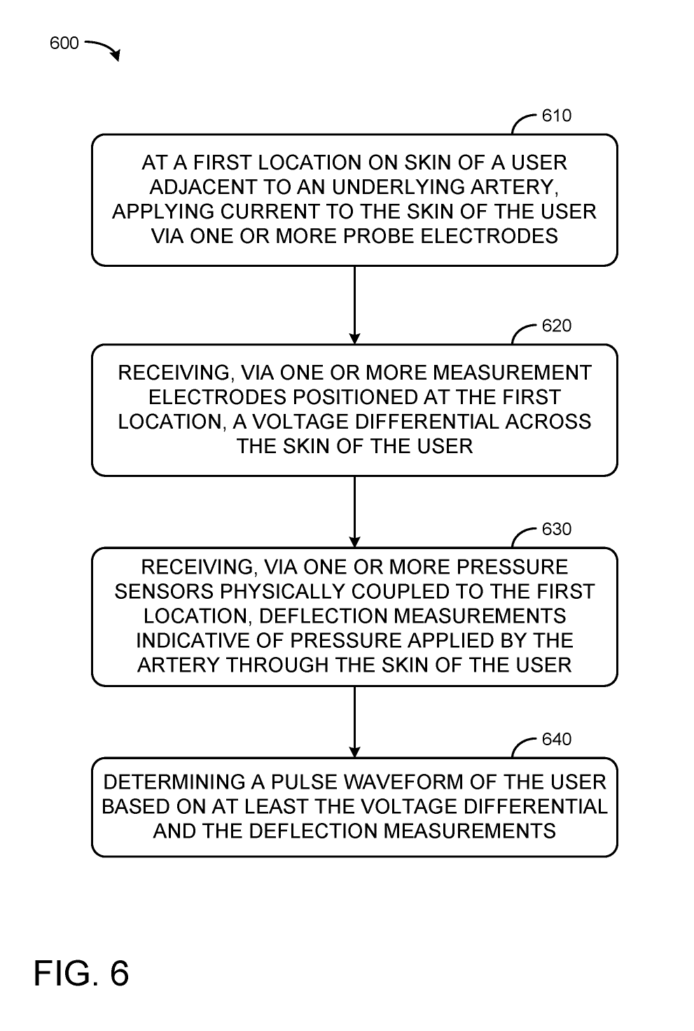

FIG. 6 shows a method 600 for determining an arterial pulse wave of a user. For example, method 600 may be used to determine an arterial pulse wave of a user based on pulse-pressure and bioimpedance measurements using a wearable system, such as wearable system 400.

At 610, method 600 includes, at a first location on skin of a user adjacent to an underlying artery, applying current to the skin of the user via one or more probe electrodes. The first location may include an area of the interfacing side of a pressure transducing pad, and a width of the interfacing side of the pressure pad may be less than a width of an arterial lumen of the user. Applying current to the skin of the user may include applying AC current to the skin of the user via one or more probe electrodes.

At 620, method 600 includes receiving, via one or more measurement electrodes positioned at the first location, a voltage differential across the skin of the user. At 630, method 600 includes receiving, via one or more pressure sensors physically coupled to the first location, deflection measurements indicative of pressure applied by the artery through the skin of the user. The one or more pressure sensors may be physically coupled to the first location via a pressure transducing pad, and the one or more probe electrodes and one or more measurement electrodes may protrude outwards from an interfacing side of the pressure transducing pad.

At 640, method 600 includes determining a pulse waveform of the user based on at least the voltage differential and the deflection measurements. In some examples, method 600 may further include indicating arterial compliance and/or stiffness of the artery based on at least the voltage differential and the deflection measurements.

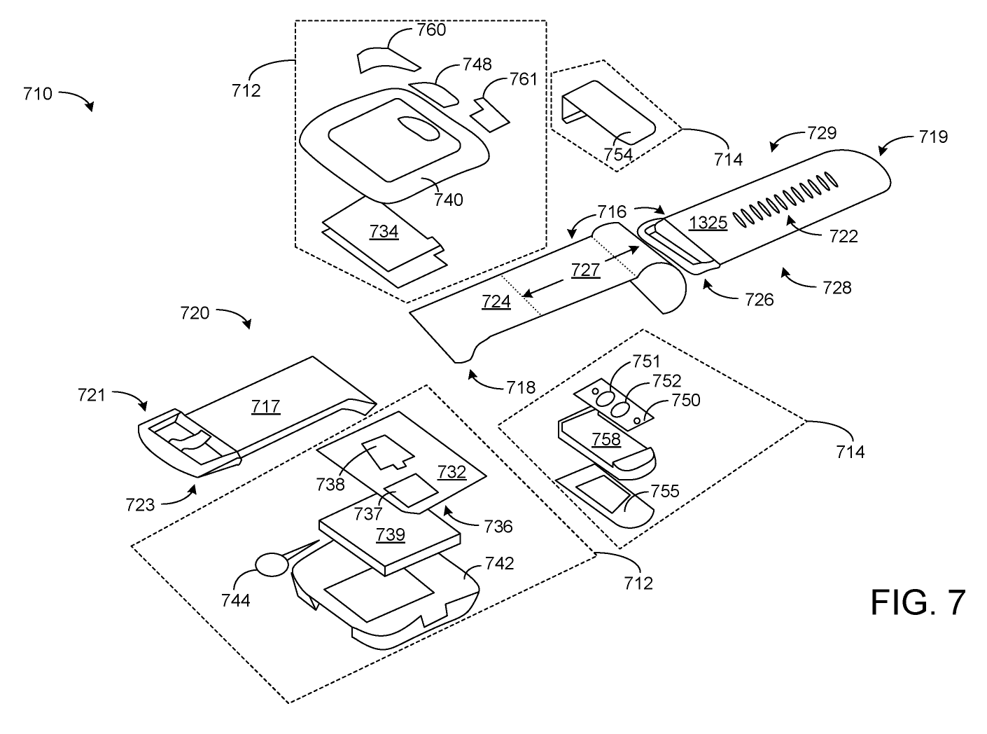

FIG. 7 shows aspects of an example sensor-and-logic system in the form of a wearable electronic device 710. The wearable electronic device 710 may be configured to measure, analyze, and/or report one or more health/fitness parameters of a wearer of wearable electronic device 710. Wearable electronic device 710 is not limiting. One or more of the features described below with reference to wearable electronic device 710 may be implemented in another sensor-and-logic system, which optionally may have a form factor that differs from wearable electronic device 710.

Wearable electronic device 710 is shown disassembled in order to depict inner componentry. The illustrated device is band-shaped and may be worn around a wrist. Wearable electronic device 710 includes a primary device 712 and a satellite device 714. Components of primary device 712 and satellite device 714 are indicated by dashed outlines. Primary device 712 may have a form function similar to the main body of a watch, and may comprise the primary user interface componentry (e.g., display, inputs, etc.) for wearable electronic device 710. Satellite device 714 may comprise pulse waveform detection componentry that may enable wearable electronic device 710 to function as a wearable cardiovascular monitoring device. The accuracy of pulse waveform detection may be dependent on the placement of the detection componentry relative to the wearer's skin and underlying tissue and vasculature. For example, including the pulse waveform detection componentry in satellite device 714 may enable pulse waveform detection at the underside of the wearer's wrist while primary device 712 is situated on the back of the wearer's wrist in a position that is familiar to watch-wearers. In this configuration, satellite device 714 and its internal components, including a pressure transducer assembly and a bioimpedance measurement assembly may be the functional equivalent of satellite housing 405 described with reference to FIGS. 4A-4B, while primary device 712 may be the functional equivalent of primary device 430 described with reference to FIG. 4B.

Wearable electronic device 710 is shown having a first strap 716 and a second strap 717. However, in some examples a single strap may be included, and in some examples, more than two straps may be included. The straps of wearable electronic device 710 may be elastomeric in some examples, and one or more of the straps optionally may be comprised of a conductive elastomer. First strap 716 may be connected to primary device 712 at first end 718, while second end 719 is located on the opposite, distal end of first strap 716. Similarly, second strap 717 may be connected to primary device 712 at first end 720, while second end 721 is located on the opposite, distal end of second strap 717. First strap 716 comprises primary fastening componentry 722 located towards second end 719, while second strap 717 comprises secondary fastening componentry 723 located towards second end 721. The straps and fastening componentry enable wearable electronic device 710 to be closed into a loop and to be worn on a wearer's wrist.

In this example, first strap 716 comprises a proximal portion 724 which connects to primary device 712 and a distal portion 725 that comprises primary fastening componentry 722. Proximal portion 724 and distal portion 725 may be coupled together via tertiary fastening componentry 726. In this way the distance between primary device 712 and primary fastening componentry 722 may be adjusted. However, in other examples, first strap 716 may be a single continuous strap that both connects to primary device 712 and comprises primary fastening componentry 722.

Satellite device 714 may be attached to first strap 716 at a fixed position within attachment region 727 of first strap 716, thus establishing a fixed distance between primary device 712 and satellite device 714. Primary fastening componentry 722 and secondary fastening componentry 723 are complementary, and thus may be adjustably engaged to adjust the circumference of wearable electronic device 710 without moving the fixed position of satellite device 714 relative to primary device 712. In this example, primary fastening componentry 722 includes discrete locations for engaging with secondary fastening componentry 723. However, in other examples, primary fastening componentry 722 and secondary fastening componentry 723 may be adjustably engaged along a continuous region.

Wearable electronic device 710 comprises a user-adjacent side 728 and an externally-facing side 729. As such, primary device 712, satellite device 714, first strap 716, and second strap 717 may each have a user-adjacent side and externally facing side. In the closed conformation, wearable electronic device 710 thus comprises an inner surface (user-adjacent) and an outer surface (externally facing).

Wearable electronic device 710 includes various functional components integrated into primary device 712. In particular, primary device 712 includes a compute system 732, display 734, communication suite 736, and various sensors. These components draw power from one or more energy-storage cells 739. A battery—e.g., a lithium ion battery—is one type of energy-storage cell suitable for this purpose. Examples of alternative energy-storage cells include super- and ultra-capacitors. In wearable electronic devices worn on the wearer's wrist, the energy-storage cells may be curved to fit the wrist.

In general, energy-storage cells 739 may be replaceable and/or rechargeable. In some examples, recharge power may be provided through a universal serial bus (USB) port, which may include a magnetic latch to releasably secure a complementary USB connector. In other examples, the energy-storage cells 739 may be recharged by wireless inductive or ambient-light charging. In still other examples, the wearable electronic device 710 may include electro-mechanical componentry to recharge the energy-storage cells 739 from the wearer's adventitious or purposeful body motion. For example, batteries or capacitors may be charged via an electromechanical generator integrated into wearable electronic device 710. The generator may be turned by a mechanical armature that turns while the wearer is moving and wearing wearable electronic device 710.

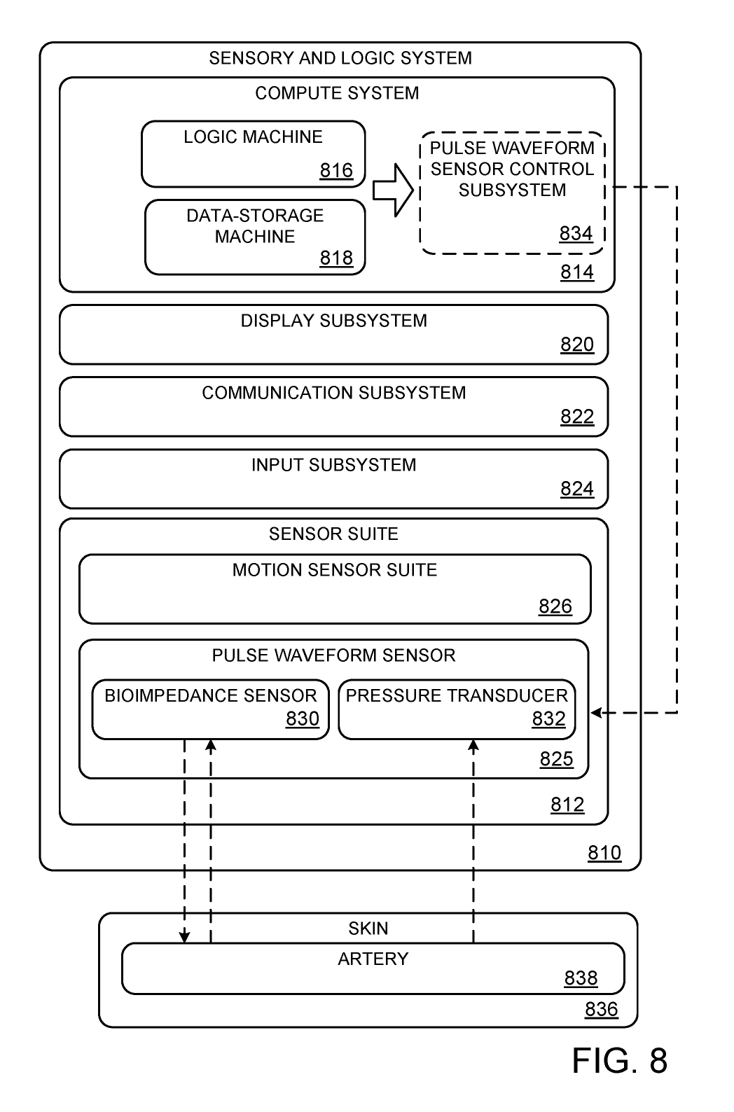

Within primary device 712, compute system 732 is situated below display 734 and operatively coupled to display 734, along with communication suite 736, and various sensors. The compute system 732 includes a data-storage machine 737 to hold data and instructions, and a logic machine 738 to execute the instructions. Aspects of compute system 732 are described in further detail with reference to FIG. 8. These components may be situated within primary device 712 between top device housing frame 740 and bottom device housing frame 742. Primary device 712 may further comprise other actuators that may be utilized to communicate with the wearer, such as haptic motor 744, and/or a loudspeaker (not shown).

Display 734 may be any suitable type of display. In some configurations, a thin, low-power light emitting diode (LED) array or a liquid-crystal display (LCD) array may be used. An LCD array may be backlit in some implementations. In other implementations, a reflective LCD array (e.g., a liquid crystal on silicon, (LCOS) array) may be frontlit via ambient light. A curved display may also be used. Further, active-matrix organic light-emitting diode (AMOLED) displays or quantum dot displays may be used.

Communication suite 736 may include any appropriate wired or wireless communication componentry. In some examples, the communication suite 736 may include a USB port, which may be used for exchanging data between wearable electronic device 710 and other computer systems, as well as providing recharge power. The communication suite 736 may further include two-way Bluetooth, Wi-Fi, cellular, near-field communication and/or other radios. In some implementations, communication suite 736 may include an additional transceiver for optical (e.g., infrared) communication.

In wearable electronic device 710, a touch-screen sensor may be coupled to display 734 and configured to receive touch input from the wearer. The touch-screen sensor may be resistive, capacitive, or optically based. Pushbutton sensors may be used to detect the state of push button 748, which may include rockers. Input from the pushbutton sensor may be used to enact a home-key or on-off feature, control audio volume, turn a microphone on or off, etc.

Wearable electronic device 710 may include a plurality of additional sensors. Such sensors may include one or more microphones, visible-light sensors, ultraviolet sensors, and/or ambient temperature sensors. A microphone may provide input to compute system 732 that may be used to measure the ambient sound level or receive voice commands from the wearer. Input from the visible-light sensor, ultraviolet sensor, and ambient temperature sensor may be used to assess aspects of the wearer's environment—i.e., the temperature, overall lighting level, and whether the wearer is indoors or outdoors.

A secondary compute system 750 is located within satellite device 714. Secondary compute system 750 may include a data-storage machine 751 to hold data and instructions, and a logic machine 752 to execute the instructions. Secondary compute system 750 may be situated between top satellite housing frame 754 and bottom satellite housing frame 755. Top satellite housing frame 754 and bottom satellite housing frame 755 may be configured to couple satellite device 714 to a fixed position within attachment region 727 on first strap 716 through the use of screws, bolts, clamps, etc. Top satellite housing frame 754 and bottom satellite housing frame 755 are shown as separate components, but in some examples, they may be coupled together by a hinge on one end, allowing satellite device 714 to be latched together around first strap 716 at the other end.

Secondary compute system 750 may be communicatively coupled to compute system 732. Satellite device 714 may mediate communication between secondary compute system 750 and compute system 732. For example, satellite device 714 may include one or more conductive contacts configured to physically intersect with one or more conductive wires extending from primary device 712 through attachment region 727 within first strap 716. In other examples, secondary compute system 750 may be coupled to compute system 732 via capacitive contact between one or more conductive contacts on satellite device 714 and one or more conductive wires within first strap 716. In other examples, a ribbon cable may extend from primary device 712 through first strap 716 such that one or more contacts on satellite device 714 can intersect with the ribbon cable when the satellite device 714 is affixed to first strap 716. In some examples, secondary compute system 750 may communicate with compute system 732 via wireless communication. In some examples, satellite device 714 may include one or more energy-storage cells. In other examples, satellite device 714 and components housed therein may draw power from energy-storage cells 739.

A bioimpedance measurement device 758 is located within satellite device 714. When placed above the wearer's radial artery, the bioimpedance measurement device 758 may determine a voltage differential present at the radial artery. Bioimpedance measurement device 758 may comprise a plurality of electrodes configured to interface with the skin of a user, including electrodes configured to apply a current to the skin of the user, and electrodes configured to measure a voltage differential across the skin of the user.

The determined voltage differential over time may then be converted into pulse waveform signals and utilized to determine the wearer's heart rate, blood pressure, and other cardiovascular properties. In some examples, a pulse pressure device (not shown) may be located within satellite device 714 in addition to bioimpedance measurement device 758. Attachment region 727 may comprise a plurality of possible sensing locations, each possible sensing location having a different effective distance from primary device 712 along the first strap 716. In some examples, attachment region 727 may comprise a plurality of continuous possible sensing locations, while in other examples attachment region 727 may comprise a plurality of discrete possible sensing locations. By adjusting the distance between primary device 712 and satellite device 714, satellite device 714 and bioimpedance measurement device 758 may be placed directly over the wearer's radial artery while primary device 712 is positioned on the back of the wearer's wrist. In some examples, satellite device 714 may be coupled to first strap 716 at a fixed position (e.g., at second end 719). In such examples, the distance between satellite device 714 and primary device 712 may be adjusted via interactions between satellite device 714 and first strap 716, via interactions between first strap 716 and primary device 712, and/or between regions of first strap 716.

Bottom satellite housing frame 755 is shown with an opening through which bioimpedance measurement device 758 can establish contact with the wearer's wrist at the radial artery. However, in some examples, electrodes of bioimpedance measurement device may be situated on an external surface of bottom satellite housing frame 755. Wearable electronic device 710 may be configured to instruct the wearer to adjust the position of satellite device 714 relative to the radial artery if a signal quality of the measured bioimpedance is below a threshold. In some examples, wearable electronic device 710 may be configured to self-adjust the position of satellite device 714 and/or the overall circumference of wearable electronic device 710.

In some examples, bioimpedance measurement device 758 may be housed and configured to interface with a wearer's wrist independently from primary device 712. For example, bioimpedance measurement device 758 may be worn on one wrist, while primary device 712 may be worn on the other wrist. In other examples, bioimpedance measurement device 758 may be configured to be worn while primary device 712 is not worn. Bioimpedance measurement device 758 may thus be configured to communicate with one or more additional computing devices, (e.g., via secondary compute system 750) such as a personal computer, tablet computer, smart phone, smart watch, gaming device, etc. The bioimpedance measurement electronics may be housed either internal to or external to the bioimpedance measurement device 758. For example, bioimpedance measurement electronics may be housed within primary device 712, while bioimpedance electrodes are housed within satellite device 714.

FIG. 7 shows a pair of contact sensor modules 760 and 761 situated on top device housing frame 740, which may be touchable by a wearer using fingers on the hand opposite the wrist where wearable electronic device 710 is worn. In some examples, other contact sensor modules may be included in addition to or as an alternative to contact sensor modules 760 and 761. As one example, other contact modules may be attached to user-adjacent side 728 of primary device 712, first strap 716 and/or second strap 717, and thus be held in contact with points on the wearer's wrist when wearable electronic device 710 is worn. As another example, one or more contact modules may be situated at or near secondary fastening componentry 723 on the externally-facing side 729 of wearable electronic device 710 when wearable electronic device 710 is closed into a loop, thus allowing the wearer to contact a point on their body reachable with the underside of the wearer's wrist. Additionally or alternatively, one or more contact modules may be situated on the externally-facing side 729 of the loop at first strap 716 and/or second strap 717.

Contact sensor modules 760 and 761 may include independent or cooperating sensor elements, to provide a plurality of sensory functions. For example, contact sensor modules 760 and 761 may provide an electrical resistance and/or capacitance sensory function, which measures the electrical resistance and/or capacitance of the wearer's skin. Compute system 732 may use such input to assess whether or not the device is being worn, for instance. In some implementations, the sensory function may be used to determine how tightly wearable electronic device 710 is being worn. In some examples, a contact sensor module may also provide measurement of the wearer's skin temperature. In some examples, contacting multiple contact sensor modules may allow compute system 732 to determine an electrocardiogram (EKG) of the wearer.

Wearable electronic device 710 may also include motion sensing componentry, such as an accelerometer, gyroscope, and magnetometer. The accelerometer and gyroscope may furnish acceleration data along three orthogonal axes as well as rotational data about the three axes, for a combined six degrees of freedom. This sensory data can be used to provide a pedometer/calorie-counting function, for example. Data from the accelerometer and gyroscope may be combined with geomagnetic data from the magnetometer to further define the inertial and rotational data in terms of geographic orientation. The wearable electronic device 710 may also include a global positioning system (GPS) receiver for determining the wearer's geographic location and/or velocity. In some configurations, the antenna of the GPS receiver may be relatively flexible and extend into straps 716 and/or 717. In some examples, data from the motion sensing componentry may be utilized to determine a position of the wearable electronic device 710, contact sensor modules 760 and or 761, and/or bioimpedance measurement device 758 relative to predetermined sensing locations on the body of the device wearer.

In some examples, wearable electronic device 710 may also include one or more optical sensors paired with one or more optical sources. The optical sources may be configured to illuminate the skin and/or the underlying tissue and blood vessels of the wearer, while the optical sensors may be configured to detect illumination reflected off of the skin and/or the underlying tissue and blood vessels of the wearer. This optical data may be communicated to compute system 732, where the data may be used to determine the wearer's blood-oxygen level, pulse, blood glucose levels, or other biometric markers with optical signatures.

Compute system 732, via the sensory functions described herein, is configured to acquire various forms of information about the wearer of wearable electronic device 710. Such information must be acquired and used with utmost respect for the wearer's privacy. Accordingly, the sensory functions may be enacted subject to opt-in participation of the wearer. In implementations where personal data is collected on the device and transmitted to a remote system for processing, that data may be anonymized. In other examples, personal data may be confined to the wearable electronic device, and only non-personal, summary data transmitted to the remote system.

As evident from the foregoing description, the methods and processes described herein may be tied to a sensory-and-logic system of one or more machines. Such methods and processes may be implemented as a computer-application program or service, an application-programming interface (API), a library, firmware, and/or other computer-program product. FIG. 7 shows one, non-limiting example of a sensory-and-logic system to enact the methods and processes described herein. However, these methods and process may also be enacted on sensory-and-logic systems of other configurations and form factors, as shown schematically in FIG. 8.

FIG. 8 schematically shows a form-agnostic sensory-and-logic system 810 that includes a sensor suite 812 operatively coupled to a compute system 814. The compute system includes a logic machine 816 and a data-storage machine 818. The compute system is operatively coupled to a display subsystem 820, a communication subsystem 822, an input subsystem 824, and/or other components not shown in FIG. 8.

Logic machine 816 includes one or more physical devices configured to execute instructions. The logic machine may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

Logic machine 816 may include one or more processors configured to execute software instructions. Additionally or alternatively, the logic machine may include one or more hardware or firmware logic machines configured to execute hardware or firmware instructions. Processors of the logic machine may be single-core or multi-core, and the instructions executed thereon may be configured for sequential, parallel, and/or distributed processing. Individual components of a logic machine optionally may be distributed among two or more separate devices, which may be remotely located and/or configured for coordinated processing. Aspects of a logic machine may be virtualized and executed by remotely accessible, networked computing devices in a cloud-computing configuration.

Data-storage machine 818 includes one or more physical devices configured to hold instructions executable by logic machine 816 to implement the methods and processes described herein. When such methods and processes are implemented, the state of the data-storage machine may be transformed—e.g., to hold different data. The data-storage machine may include removable and/or built-in devices; it may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. The data-storage machine may include volatile, nonvolatile, dynamic, static, read/write, read-only, random-access, sequential-access, location-addressable, file-addressable, and/or content-addressable devices.

Data-storage machine 818 includes one or more physical devices. However, aspects of the instructions described herein alternatively may be propagated by a communication medium (e.g., an electromagnetic signal, an optical signal, etc.) that is not held by a physical device for a finite duration.

Aspects of logic machine 816 and data-storage machine 818 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

Display subsystem 820 may be used to present a visual representation of data held by data-storage machine 818. This visual representation may take the form of a graphical user interface (GUI). As the herein described methods and processes change the data held by the storage machine, and thus transform the state of the storage machine, the state of display subsystem 820 may likewise be transformed to visually represent changes in the underlying data. Display subsystem 820 may include one or more display subsystem devices utilizing virtually any type of technology. Such display subsystem devices may be combined with logic machine 816 and/or data-storage machine 818 in a shared enclosure, or such display subsystem devices may be peripheral display subsystem devices. Display 734 of FIG. 7 is an example of display subsystem 820.

Communication subsystem 822 may be configured to communicatively couple compute system 814 to one or more other computing devices. The communication subsystem may include wired and/or wireless communication devices compatible with one or more different communication protocols. As non-limiting examples, the communication subsystem may be configured for communication via a wireless telephone network, a local- or wide-area network, and/or the Internet. Communication suite 736 of FIG. 7 is an example of communication subsystem 822.

Input subsystem 824 may comprise or interface with one or more user-input devices such as a keyboard, touch screen, button, dial, joystick, or switch. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition. Push button 748 of FIG. 7 is an example of input subsystem 824.

Sensor suite 812 may include one or more different sensors—e.g., pulse waveform sensor 825, a touch-screen sensor, push-button sensor, microphone, visible-light sensor, ultraviolet sensor, ambient-temperature sensor, contact sensors, and/or GPS receiver—as described above with reference to FIG. 7. Sensor suite 812 may include motion sensor suite 826. Motion sensor suite 826 may include one or more of an accelerometer, gyroscope, magnetometer, or other suitable motion detectors.

As described herein, pulse waveform sensor 825 may include bioimpedance sensor 830 and/or pressure transducer 832. Compute system 814 may include pulse waveform sensor control subsystem 834, which may be communicatively coupled to logic machine 816 and data-storage machine 818. Bioimpedance sensor 830 may comprise two or more sets of electrodes; a first set configured to inject current into the skin 836 of a user, and a second set configured to transduce a voltage differential across the skin 836 of a user. The voltage differential may be indicative of volume changes of the radial artery 838 of the user. Pressure transducer 832 may comprise one or more piezo-resistive sensors configured to provide absolute pressure signals to compute system 814 via an analog-to-digital converter. Pressure transducer 832 may be configured to transduce pressure waves from the radial artery 838 through the skin 836 of the user.

Pulse waveform sensor control subsystem 834 may further process the raw signals to determine heart rate, blood pressure, caloric expenditures, etc. Processed signals may be stored and output via compute system 814. Control signals sent to pulse waveform sensor 825 may be based on signals received from bioimpedance sensor 830, pressure transducer 832, signals derived from sensor suite 812, information stored in data-storage machine 818, input received from communication subsystem 822, input received from input subsystem 824, etc.

In one example, a system for determining an arterial pulse wave of a user comprises a pressure transducing pad temporarily attachable to skin of a user, the pressure transducing pad configured to deflect outwards from the skin proportionate to pressure applied by an artery; a sensor configured to measure outward deflection of the pressure transducing pad; and a plurality of electrodes coupled to the pressure transducing pad and configured to interface with the skin of the user when the pressure transducing pad is attached to the skin of the user, the plurality of electrodes comprising electrodes configured to apply a current to the skin of the user, and electrodes configured to measure a voltage differential across the skin of the user. In such an example, or any other example, the plurality of electrodes may additionally or alternatively comprise two electrodes configured to apply a current to the skin of the user, and further comprise two electrodes configured to measure a voltage differential across the skin of the user. In any of the preceding examples, or any other example, the plurality of electrodes may additionally or alternatively be thin strip electrodes arranged in parallel such that the two electrodes configured to apply a current to the skin of the user are outside of the two electrodes configured to measure a voltage differential across the skin of the user. In any of the preceding examples, or any other example, the plurality of electrodes may additionally or alternatively be round electrodes arranged in a four-corner pattern. In any of the preceding examples, or any other example, the plurality of electrodes may additionally or alternatively occupy a footprint less than or equal to 3 cm×3 cm. In any of the preceding examples, or any other example, the plurality of electrodes may additionally or alternatively comprise two electrodes configured to both apply a current to the skin of the user and to measure a voltage differential across the skin of the user. In any of the preceding examples, or any other example, the sensor configured to measure outward deflection of the pressure transducing pad may additionally or alternatively be a piezo-electric pressure transducer. In any of the preceding examples, or any other example, the system may additionally or alternatively comprise a wearable assembly including fastening componentry configured to couple the pressure transducing pad in place at the artery. In any of the preceding examples, or any other example, the artery may additionally or alternatively be a radial artery. In any of the preceding examples, or any other example, the artery may additionally or alternatively be an ulnar artery. In any of the preceding examples, or any other example, the pressure transducing pad may additionally or alternatively include an interfacing side configured to contact the skin of the user, and wherein the plurality of electrodes protrude outward from the interfacing side of the pressure transducing pad.

In another example, a method for determining an arterial pulse wave of a user comprises, at a first location on skin of a user adjacent to an underlying artery, applying current to the skin of the user via one or more probe electrodes; receiving, via one or more measurement electrodes positioned at the first location, a voltage differential across the skin of the user; receiving, via one or more pressure sensors physically coupled to the first location, deflection measurements indicative of pressure applied by the artery through the skin of the user; determining a pulse wave of the user based on at least the voltage differential and the deflection measurements. In such an example, or any other example, the one or more pressure sensors may additionally or alternatively be physically coupled to the first location via a pressure transducing pad, and the one or more probe electrodes and one or more measurement electrodes may additionally or alternatively protrude outwards from an interfacing side of the pressure transducing pad. In any of the preceding examples, or any other example, the first location includes an area of the interfacing side of the pressure pad, and wherein a width of the interfacing side of the pressure pad is less than a width of an arterial lumen of the user. In any of the preceding examples, or any other example, the method may additionally or alternatively comprise indicating an arterial stiffness of the artery based on at least the voltage differential and the deflection measurements. In any of the preceding examples, or any other example, applying current to the skin of the user via one or more probe electrodes may additionally or alternatively include applying AC current to the skin of the user via one or more probe electrodes.

In yet another example, a wearable system for determining an arterial pulse wave of a user, comprises fastening componentry to temporarily couple the wearable system to a wrist of the user; a plurality of electrodes configured to contact the skin of the user, the plurality of electrodes occupying a footprint less than or equal to 3 cm×3 cm, the plurality of electrodes comprising electrodes configured to apply a current to the skin of the user, and electrodes configured to measure a voltage differential across the skin of the user; and a controller configured to output a pulse wave of the user based on at least the voltage differential. In such an example, or any other example, the plurality of electrodes may additionally or alternatively comprise two electrodes configured to apply a current to the skin of the user, and may additionally or alternatively comprise two electrodes configured to measure a voltage differential across the skin of the user. In any of the preceding examples, or any other example the plurality of electrodes may additionally or alternatively be thin strip electrodes arranged in parallel such that the two electrodes configured to apply a current to the skin of the user are outside of the two electrodes configured to measure a voltage differential across the skin of the user. In any of the preceding examples, or any other example the plurality of electrodes may additionally or alternatively be round electrodes arranged in a four-corner pattern.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above- described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A system for determining an arterial pulse wave of a user, comprising:
a pressure transducing pad temporarily attachable to skin of a user, the pressure transducing pad configured to deflect outwards from the skin proportionate to pressure applied by an artery;
a sensor configured to measure outward deflection of the pressure transducing pad; and
a plurality of electrodes coupled to the pressure transducing pad and configured to interface with the skin of the user when the pressure transducing pad is attached to the skin of the user, the plurality of electrodes comprising electrodes configured to apply a current to the skin of the user, and electrodes configured to measure a voltage differential across the skin of the user.

2. The system of claim 1, wherein the plurality of electrodes comprise two electrodes configured to apply a current to the skin of the user, and further comprise two electrodes configured to measure a voltage differential across the skin of the user.

3. The system of claim 2, wherein the plurality of electrodes are thin strip electrodes arranged in parallel such that the two electrodes configured to apply a current to the skin of the user are outside of the two electrodes configured to measure a voltage differential across the skin of the user.

4. The system of claim 2, wherein the plurality of electrodes are round electrodes arranged in a four-corner pattern.

5. The system of claim 2, wherein the plurality of electrodes occupy a footprint less than or equal to 3 cm x 3 cm.

6. The system of claim 1, wherein the plurality of electrodes comprise two electrodes configured to both apply a current to the skin of the user and to measure a voltage differential across the skin of the user.

7. The system of claim 1, wherein the sensor configured to measure outward deflection of the pressure transducing pad is a piezo-electric pressure transducer.

8. The system of claim 1, further comprising a wearable assembly including fastening componentry configured to couple the pressure transducing pad in place at the artery.

9. The system of claim 8, wherein the artery is a radial artery.

10. The system of claim 8, wherein the artery is an ulnar artery.

11. The system of claim 1, wherein the pressure transducing pad includes an interfacing side configured to contact the skin of the user, and wherein the plurality of electrodes protrude outward from the interfacing side of the pressure transducing pad.

12. A method for determining an arterial pulse wave of a user, comprising:
at a first location on skin of a user adjacent to an underlying artery, applying current to the skin of the user via one or more probe electrodes;
receiving, via one or more measurement electrodes positioned at the first location, a voltage differential across the skin of the user;
receiving, via one or more pressure sensors physically coupled to the first location, deflection measurements indicative of pressure applied by the artery through the skin of the user;
determining a pulse wave of the user based on at least the voltage differential and the deflection measurements.

13. The method of claim 12, wherein the one or more pressure sensors are physically coupled to the first location via a pressure transducing pad, and wherein the one or more probe electrodes and one or more measurement electrodes protrude outwards from an interfacing side of the pressure transducing pad.

14. The method of claim 13, wherein the first location includes an area of the interfacing side of the pressure pad, and wherein a width of the interfacing side of the pressure pad is less than a width of an arterial lumen of the user.

15. The method of claim 12, further comprising:
indicating an arterial stiffness of the artery based on at least the voltage differential and the deflection measurements.

16. The method of claim 12, wherein applying current to the skin of the user via one or more probe electrodes includes applying AC current to the skin of the user via one or more probe electrodes.

17. A wearable system for determining an arterial pulse wave of a user, comprising:
fastening componentry to temporarily couple the wearable system to a wrist of the user;
a plurality of electrodes configured to contact the skin of the user, the plurality of electrodes occupying a footprint less than or equal to 3 cm×3 cm, the plurality of electrodes comprising electrodes configured to apply a current to the skin of the user, and electrodes configured to measure a voltage differential across the skin of the user; and
a controller configured to output a pulse wave of the user based on at least the voltage differential.

18. The wearable system of claim 17, wherein the plurality of electrodes comprise two electrodes configured to apply a current to the skin of the user, and further comprise two electrodes configured to measure a voltage differential across the skin of the user.

19. The wearable system of claim 18, wherein the plurality of electrodes are thin strip electrodes arranged in parallel such that the two electrodes configured to apply a current to the skin of the user are outside of the two electrodes configured to measure a voltage differential across the skin of the user.

20. The wearable system of claim 18, wherein the plurality of electrodes are round electrodes arranged in a four-corner pattern.
